(19) United States
(12) Patent Application Publication
(10) Pub. No.: US 2020/0078537 A1
(43) Pub. Date: Mar. 12, 2020

(54) ATOMIZER DRUG DELIVERY NOZZLE, AND INTELLIGENTLY SELF-ADJUSTABLE ATOMIZER DRUG DELIVERY APPARATUS AND METHOD FOR USING SAME

(71) Applicant: QINGDAO FUTURE MEDICAL TECHNOLOGY CO., LTD., QNGDAO, Shandong Province (CN)

(72) Inventor: WEILAI WANG, QINGDAO, SHANDONG PROVINCE (CN)

(21) Appl. No.: 16/468,629

(22) PCT Filed: Dec. 30, 2016
(86) PCT No.: PCT/CN2016/113314

§ 371 Date: Jun. 11, 2019

(30) Foreign Application Priority Data
Dec. 12, 2016 (CN) 201611137792.2
Dec. 12, 2016 (CN) 201621355870.1

(51) Int. Cl. A61M 11/04; A61M 39/24

(52) U.S. Cl. CPC A61M 11/042; A61M 39/24; A61M 2230/42; A61M 2205/0288; A61M 2205/50; A61M 2205/3368; A61M 2205/18; A61M 2205/581; A61M 2205/3317

(57) ABSTRACT

Disclosed is an atomizer drug delivery nozzle, and an intelligently self-adjustable atomizer drug delivery apparatus and a method for using same, characterized in that: a nozzle body (2) is provided with an exhalation detection valve (3) unidirectionally opening toward the outside of a nozzle and an inhalation detection valve (4) unidirectionally opening toward the inside of the nozzle, wherein bodies (3.2, 4.2) of the two valves (3, 4) are respectively provided with a first magnet (3.3) and a second magnet (4.3); a tube wall of the part of the nozzle body (2) that is close to the first magnet (3.3) is provided with a first sensor (3.1), and a tube wall of the part of the nozzle body (2) that is close to the second magnet (4.3) is provided with a second sensor (4.1); the first sensor (3.1) and the second sensor (4.1) are respectively connected to an atomizer control system; and the atomizer control system comprises an intelligent module for detecting a valve state during exhalation and inhalation, and controls a working state of the atomizer according to a received valve opening state detection signal sent by the first sensor (3.1) and the second sensor (4.1). By precisely sensing a patient's breathing action in real time, the atomizer nozzle can realize intelligent self-adjustment, so that the atomizer can be used once opened, has a simplified structure, decreased production costs and a reduced electricity consumption.

### TECHNICAL FIELD

The invention belongs to the technical field of medical equipment, and relates to an improvement of a nebulizer treatment device, and in particular to a nebulizer drug delivery nozzle and an intelligent self-adjusting nebulizer drug delivery device and a method for using the same.

### BACKGROUND

Due to the increasing global climate change and air pollution, the prevalence of asthma went up significantly year by year, so does the asthma attack on individuals, especially among the group of children from the age of three to fourteen, the asthma rate is up to 3%. Asthma does not only affect children's physical development, but also acts as an invisible “killer” that endangers children's live. Hence, many parents have concerns about the treatment of childhood asthma. Viral infection is an important factor causing wheezing and asthma attacks on children. It is noted that about 20% babies may have wheezing symptoms due to viral infection during their infancy and 16% of them may develop to asthma in case of no proper treatment. We discover that if a 6-month baby infected by a virus manifesting a repeated wheezing symptom, the breath difficulty probably lasts until the age of seven or eight and some of them would develop to childhood asthma. Respiratory virus infection is also an important factor causing asthma recurrence and asthma exacerbation. In a case-control study with 84 patients of asthma exacerbations, the viral infection rate is up to 44%.

Aerosol therapy is the globally recognized best medical treatment employed to ease different forms of asthmatic diseases, in which a nebulizer is utilized to change medications from a liquid state to a mist that is inhaled into lungs via mouth or nose. The therapeutic effect is reinforced by a proper local drug concentration. The aerosol therapy has features as being quick in taking effect, safe and convenient.

In most developed countries such as in Europe and in the United States, nebulizers have become an essential household medical tool for coping with various respiratory disease attacks, such as colds, fever, cough, throat pain, rhinitis, chronic pharyngitis, tonsillitis, asthma, obstructive (spastic) bronchitis, emphysema, chronic bronchitis, bronchiectasis, mucoviscidosis, cystic pulmonary fibrosis and situations that patients needed to humidify airway or dilute sputum. In addition, according to an online sales data in China during the past two years, the market of household nebulizer has become a hotspot. With the wide use of household nebulizer, the market prospect is very promising.

Although the household nebulizers have different structures, all of those have a same problem that the administration efficiency would be affected more or less by the user breathing pattern, which is especially obvious in using an atomizer jet while not evident in using an ultrasonic device or a vibrating screen device, and leads to inter-individual and intra-individual differences. Therefore, a good nebulizer should be able to adjust its atomization process and perform intelligently according to the user's respiratory condition, such as the AAD (Adaptive Aerosol Delivery), namely self-adjusting nebulizer drug delivery device, which is exemplified by the Prodose AAD launched in 2002 and the I-neb AAD (Philips Respironics) fusing with vibrating screen technology of OMRON™ launched in 2004.

### Technical Problem

However, the well-known AAD devices still have the following problems and deficiencies:

1. The well-known AAD device is complicated and expensive. Taking the I-neb AAD as an example, a built-in pressure sensor is used to monitor airflow pressure for obtaining an air pressure waveform. During its continuous working it is determined as the start of an inhalation if the air pressure keeps rising till the growth is greater than a preset value, or it is determined as the start of an exhalation if the air pressure keeps dropping till the decrease is greater than a preset value. It also needs to identify at least three peak values of airflow to determine the atomization interval. After the initial process, every breath action of the user is monitored to modify the time interval so as to accommodate the breathing mode in each treatment cycle. It is obvious that the whole process is demanding, especially for sensor, and the accuracy is mainly based on the sampling and prediction algorithm, which may further exert negative effect on the treatment efficiency.
2. The nebulizer could not use in an out-of-the-box mode. Because the breathing mode is determined according to the sampled airflow peak, it is necessary to sample the inhalation and exhalation actions beforehand, which is not convenient in using.
3. The complexity of the well-know AAD device leads to an increase in power consumption, which is fatal for portable devices with limited power capacity.
4. The AAD device could not control the amount of the medicine inhaled. Although the AAD device greatly improves the administration efficiency of nebulizer, it is still not able to control the dosage in an accurate way. There is a need for a device capable of avoiding overdose or deficiency of medication.

### SUMMARY OF THE INVENTION

The present invention overcomes the aforementioned limitations of nebulizer nozzle in the prior art.

The nebulizer drug delivery nozzle could accurately determine user breathing pattern in a convenient way with a detection device low in cost and power consumption so as to solve the problem that the nebulizer could not use in an out-of-the-box mode of the traditional AAD nebulizer; additionally it could avoid the situation of overdose or deficiency, and further realizing an intelligent self-adjusting control of nebulizer nozzle.

The object of the invention is achieved by the following technical solutions:

A nebulizer drug delivery nozzle including a nozzle body in a shape of tubular with one end forming a medication inlet and the other end forming a spray port, wherein includes: an exhalation detection valve arranged on the tubular wall of the nozzle body, which is configured to open one-way outwards as breathing out air to the spray port, including an exhalation detection valve body provided with a first magnet, an inhalation detection valve arranged on the tubular wall of the nozzle body, which is configured to open one-way inwards as breathing air in from the spray port, including an inhalation detection valve body provided with a second magnet; a first sensor configured to detect the state of the exhalation detection valve, which is disposed on the wall of the nozzle body adjacent to the first magnet; a second sensor configured to detect the state of the inhalation detection valve, which is disposed on the wall of the nozzle body adjacent to the second magnet; and a nebulizer control system coupled to the first sensor and the second sensor including an intellectual detection module configured to detect the state of the exhalation detection valve and the inhalation detection valve, which is configured to control an atomizer according to the valve state signal sent by the first sensor and the second sensor.

Further, the inhalation detection valve is disposed at the bottom of the tubular wall of the nozzle body, in which the inhalation detection valve body opens upwards as breathing air in from the spray port; the exhalation detection valve is disposed at the top of the tubular wall of the nozzle body, in which the exhalation detection body opens upwards as breathing out air to the spray port.

Further, the first sensor is a first reed switch and the second sensor is a second reed switch; if the exhalation detection valve body is in an open state, the magnetic sensitive contacts of the first reed switch separate; if the inhalation detection valve body is in a close state, the magnetic sensitive contacts of the second reed switch is being pulled together; if the inhalation detection valve body is in an open state, the magnetic sensitive contacts of the second reed switch separate, the atomizer is being controlled by the nebulizer control system to start to release medication; if the inhalation detection valve body is in a close state, the magnetic sensitive contacts of the second reed switch is being controlled by the nebulizer control system to stop working.

Further, the first sensor is a first Hall switch and the second sensor is a second Hall switch; the nebulizer control system includes a calculation logic module configured to output the opening angle of the exhalation detection valve and the inhalation detection valve; the nebulizer control system determines the state of the exhalation detection valve body and its opening angle according to the varied magnetic flux of the first Hall Switch; if the exhalation detection valve body is in a close state, the magnetic flux of the first Hall switch is maximum; if the exhalation detection valve body is in a full open state, the magnetic flux of the first Hall switch is minimum; the nebulizer control system determines the state of the inhalation detection valve body and its opening angle according to the varied magnetic flux of the second Hall switch; if the inhalation detection valve body is in a full open state, the magnetic flux of the second Hall switch is minimum, the atomizer is being controlled by the nebulizer control system to start to release medication; if the inhalation detection valve body is in a close state, the magnetic flux of the second Hall switch is maximum, the atomizer is being controlled by the nebulizer control system to stop working.

Further, a nebulizer drug delivery nozzle including a nozzle body in a shape of tubular with one end forming a medication inlet and the other end forming a spray port, wherein includes: an aerosol container provided with a drug inlet and an aerosol container valve, which is a solenoid valve and is connected to the medication inlet of the nozzle body; a drug concentration sensor disposed in the aerosol container; a nebulizer control system coupled to the aerosol container valve and the drug concentration sensor, which includes an intelligent detection module configured to detect the state of the exhalation detection valve and the inhalation detection valve, a calculation logic module configured to calculate the valve body opening angle and a drug concentration detection module configured to detect the medication concentration within medication container; an exhalation detection valve disposed at the top of the tubular wall of the nozzle body, which is configured to open upwards as breathing out air to the spray port, including an exhalation detection valve body provided with a first magnet; an inhalation detection valve disposed at the bottom of the tubular wall of the nozzle body, which is configured to open upwards as breathing air in from the spray port, including an inhalation detection valve body provided with a second magnet; a first Hall switch disposed on the tubular wall of the nozzle body adjacent to the first magnet, which is coupled to the nebulizer control system; a second Hall switch disposed on the bottom of the aerosol container adjacent to the second magnet, which is coupled to the nebulizer control system; wherein the nebulizer control system determines the state of the exhalation detection valve body and its opening angle according to the varied magnetic flux of the first Hall Switch; if the exhalation detection valve body is in a close state, the magnetic flux of the first Hall switch is maximum; if the exhalation detection valve body is in a full open state, the magnetic flux of the first Hall switch is minimum; the nebulizer control system determines the state of the inhalation detection valve body and its opening angle according to the varied magnetic flux of the second Hall switch; if the inhalation detection valve body is in a full open state, the magnetic flux of the second Hall switch is minimum, the atomizer is being controlled by the nebulizer control system to start to release medication; if the inhalation detection valve body is in a close state, the magnetic flux of the second Hall switch is maximum, the atomizer is being controlled by the nebulizer control system to stop working.

The nebulizer drug delivery nozzle further comprises a power module, a nebulizer connection port and a built-in or external user interface.

The nebulizer drug delivery nozzle further comprises an inner medication container provided with an inner medication container valve and a nebulizer connection port configured to connect to the drug inlet of the aerosol container; a temperature sensor disposed on the nozzle body or within the inner medication container which is coupled to the nebulizer control system; an electric heater disposed on the nozzle body or within the inner medication container which is coupled to the nebulizer control system; the nebulizer control system further comprising a detection module configured to detect the temperature within medication container.

An intelligent self-adjusting nebulizer drug delivery device includes a liquid medication container, an atomizer and a nebulizer control system, and a nebulizer drug delivery nozzle as the above-mentioned.

The intelligent self-adjusting nebulizer drug delivery device further includes an alarm connected to the nebulizer control system; wherein if the nebulizer control system detects the exhalation detection valve and the inhalation detection valve are in open state at the same time, then closing the atomizer and activating the alarm; the nebulizer control system includes a sound recognition device, a breathing frequency detection circuit and a voice guide circuit; if the sound recognition device determines that the breathing sound is greater than a threshold, or it is determined that the breathing frequency is greater than a preset frequency, the voice guide circuit makes a sound.

A method for using the intelligent self-adjusting nebulizer drug delivery device comprises:

1) Determining whether the inhalation detection valve body in an opening state by the nebulizer control system; if the inhalation detection valve body is in an opening state, starting inhalation; determining the airflow rate according to the detected opening angle of the inhalation detection valve body; activating the atomizer by the nebulizer control system if the airflow rate reaches to a preset range;
2) Adjusting atomization parameters, such as medication volume, medication concentration and the like according to types of medication, which could be obtained by medication type detection or user input, inhalation volume and the like.

During the atomization process, if the inhalation volume does not conform certain patterns, such as the inhalation volume is much greater than the preset volume, the inhalation volume is much less than the preset volume, the inhalation volume fluctuates, the breathing frequency is higher than a preset frequency or the breathing frequency is lower than a preset frequency, the built-in or external user interface could remind the user to adjust their inhalation volume or breathing frequency; preferably, if the inhalation volume or breathing frequency maintains at an ideal level, the built-in or external user interface could reminder the user to keep their current inhalation volume or breathing frequency. In addition, it may activate some games or contests to make the user more compliant in treatment.

3) Terminating the atomization process by the nebulizer control system if the inhalation airflow rate is lower than the preset range; calculating a total inhalation volume according to the overall time of this atomization process and inhalation volume to determining whether the total inhalation volume is in a preset optimal range; outputting the result via the built-in or external user interface: such that if the total inhalation volume satisfies the treatment requirement, the built-in or external user interface output corresponding signal to remind the user keeping current state; if the total inhalation volume does not satisfy the treatment requirement, the built-in or external user interface output corresponding signal to remind the user adjusting current state.

Further, the nebulizer control system also detects a total inhalation time and a total exhalation time in one atomization process to calculation a breathing ratio, and then determining whether the breathing ratio is in a rational setting range; and outputting the result to user via the built-in or external user interface.

Optionally, after one atomization process, the nebulizer control system reminds the user to hold their breath for 5 to 10 seconds via the built-in or external user interface and monitors if the user performs corresponding action; if the user does not perform corresponding action, then reminding the user to adjust.

4) Monitoring parameters such as breathing frequency, breathing volume, total breathing volume and breathing ratio in the atomization process; if the breathing begins to present a shallow and frequent pattern, it means the inhalation volume is comparatively low, increasing the time period of atomization process to ensure treatment effect.

Further, the method for using the intelligent self-adjusting nebulizer drug delivery device further includes an adverse reaction detection step and an adverse reaction processing step:

5) If in the atomization process, the breath frequency is higher than a preset adverse reaction threshold, it could be considered as a sign of the occurrence of adverse reaction; determining whether the breathing sound is higher than a preset sound threshold via the sound recognition device of the nebulizer control system; if the breathing sound is higher than the preset sound threshold, terminating the atomization process and reminding the user to exhale forcefully.
6) Compared the exhalation volume within one second as the user to exhale forcefully with a sampled exhalation volume at the beginning of the atomization process; if the two exhalation volumes are significantly different, it is determined as the occurrence of an adverse reaction.
7) If the situations in Step 5 and Step 6 repeatedly occur, terminating the atomization process, generating an adverse reaction report and reminding the user to contact a doctor.

Further, the method for using the intelligent self-adjusting nebulizer drug delivery device further includes a precise medication dosage control step:

8) Gradually increasing the medication concentration within the aerosol container 6 and within the inner medication container 7 from the minimum to the maximum therapeutic concentration via the nebulizer control system as the atomization process starts.

9) if detecting an adverse reaction in the process as gradually increasing medication concentration, then stopping increasing the medication concentration and set the current medication concentration as an optimal concentration for the user by the nebulizer control system.

The advantages and positive effects of the present invention are:

1. In the nebulizer drug delivery nozzle, the one-way valve and corresponding detection circuits, and the nebulizer control system could achieve an accurate detection of the patient's breathing behavior, so it could fulfill an accurate adjustment in atomization process according to the patient's breathing behavior, so as to solve the problem of the AAD that the nebulizer could not able to work as an out-of-the-box device; in the meanwhile this general arrangement could reduce the complexity and cost, thereby achieving an energy-saving and environmental-friendly purpose.
2. In addition to the breath pattern determination and the implementation of the one-way valve, a breathing volume determination process is introduced to further improve the control accuracy based on the patient's breathing airflow rate; further, on the basis of the breath pattern detection and the breathing volume detection, it further discloses a detection and processing way to cope with the adverse reaction during the atomization process.
3. In addition to the accurate control of the atomization process according to the patient's airway flow rate, the atomization process is further being adjusted according to the aerosol medication concentration, so as to avoid the occurrence of adverse reaction due to high concentration and ensure the treatment effect at the same time.
4. An alarm is provided to warn the user if an adverse reaction caused by drug reaction occurs.
5. The intelligent self-adjusting nebulizer disclosed by the present invention is adaptive to those manufactured nebulizers for making it more intelligent.
6. The temperature of aerosol generated in the process of atomization is comparatively low, which may cause reactive airway enthalpy. Hence, the nebulizer drug delivery nozzle may further include a temperature sensor and an electric heater, which may be disposed on the nozzle body or within the inner medication container. The temperature sensor and the electric heater are connected to the nebulizer control system. The nebulizer control system further comprises a temperature detecting module. During the process of atomization, the nebulizer control system automatically regulates the temperature of aerosol according to the detected results so as to ensure it is be used in a comfort way.

### BRIEF DESCRIPTION OF THE DRAWING

FIG. 1 is a perspective view showing a connection structure of a nebulizer drug delivery nozzle and a liquid medication container according to one embodiment of the present invention;

FIG. 2 is a cross-sectional view showing an open state of an inhalation detection valve in a nebulizer drug delivery nozzle according to one embodiment of the present invention;

FIG. 3 is a cross-sectional view showing an open state of an exhalation detection valve in a nebulizer drug delivery nozzle according to one embodiment of the present invention;

FIG. 4 is a perspective view showing a connection of a nebulizer drug delivery nozzle including an aerosol container according to one embodiment of the present invention;

FIG. 5 is a perspective view showing a connection of a nebulizer drug delivery nozzle including an aerosol container and an inner medication container according to one embodiment of the present invention;

FIG. 6 is a functional block diagram of a control system of a nebulizer drug delivery nozzle according to one embodiment of the present invention.

Wherein, 1-liquid medication container, 2-nozzle body, 2.1-spray port, 3-exhalation detection valve, 3.1-first sensor (first reed switch or first Hall switch), 3.2-exhalation detection valve body, 3.3-first magnet, 4-inhalation detection valve, 4.1-second sensor (second reed switch or second Hall switch), 4.2-inhalation detection valve body, 4.3-second magnet, 5-atomizer, 6-aerosol container, 6.1-aerosol container valve, 7-inner medication container, 7.1-inner medication container valve.

### DETAILED DESCRIPTION

Further features of the inventive embodiments of the present invention will become apparent to those skilled in the art to which the embodiments relate from reading the specification and claims with reference to the accompanying drawings.

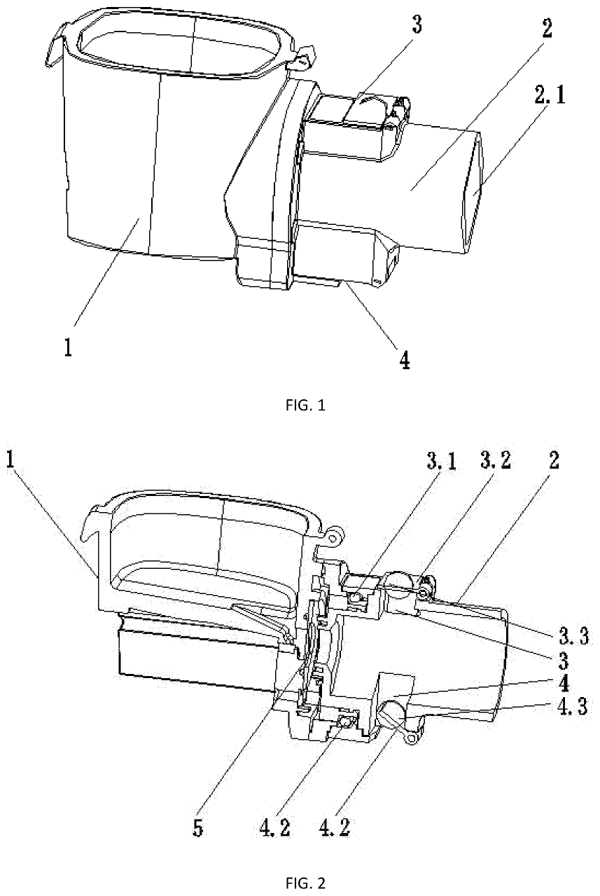

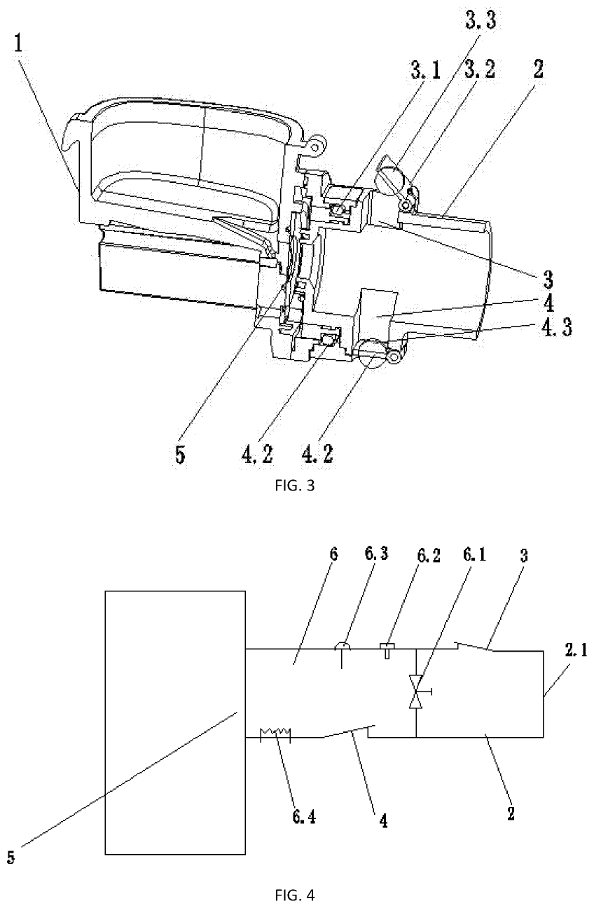

The general arrangement of a nebulizer drug delivery nozzle according to an embodiment of the present invention is depicted in FIG. 1-3 and FIG. 6. The nebulizer drug delivery nozzle includes a nozzle body 2 in a shape of tubular, wherein a medication inlet formed at one end of the nozzle body 2 and a spray port 2.1 formed at the other end of the nozzle body 2. An exhalation detection valve 3 is arranged on the tubular wall of the nozzle body 2 which is configured to open one-way outwards as breathing out air to the spray port 2.1. An inhalation detection valve 4 is also arranged on the tubular wall of the nozzle body 2 which is configured to open one-way inwards as breathing air in from the spray port 2.1. The exhalation detection valve 3 includes an exhalation detection valve body 3.2 provided with a first magnet 3.3, and the inhalation detection valve 4 includes an inhalation detection valve body 4.2 provided with a second magnet 4.3. A first sensor 3.1 configured to detect the state of the exhalation detection valve 3 is disposed on the wall of the nozzle body 2 adjacent to the first magnet 3.3, and a second sensor 4.1 configured to detect the state of the inhalation detection valve 4 is disposed on the wall of the nozzle body 2 adjacent to the second magnet 4.3. The first sensor 3.1 and the second sensor 4.1 are connected to a nebulizer control system, wherein the nebulizer control system including an intelligent detection module of the state of the exhalation detection valve and the inhalation detection valve is configured to control an atomizer 5 according to the valve state signal sent by the first sensor 3.1 and the second sensor 4.1.

The embodiment will be described in detail as follows.

Referring to FIG. 1-3 and FIG. 6, the nebulizer drug delivery nozzle according to a first present embodiment includes a nozzle body 2 in a shape of tubular, wherein a medication inlet formed at one end of the nozzle body 2 and a spray port 2.1 formed at the other end of the nozzle body 2. In this embodiment, the first sensor 3.1 is a first reed switch and the second sensor 3.2 is a second reed switch. An exhalation detection valve 3 is arranged on the tubular wall of the nozzle body 2 which is configured to open one-way outwards as breathing out air to the spray port 2.1. An inhalation detection valve 4 is also arranged on the tubular wall of the nozzle body 2 which is configured to open one-way inwards as breathing air in from the spray port 2.1. The exhalation detection valve 3 and the inhalation detection valve 4 are one-way valve. The exhalation detection valve 3 includes an exhalation detection valve body 3.2 provided with a first magnet 3.3, and the inhalation detection valve 4 includes an inhalation detection valve body 4.2 provided with a second magnet 4.3. The first reed switch 3.1 configured to detect the state of the exhalation detection valve 3 is disposed on the wall of the nozzle body 2 adjacent to the first magnet 3.3, and the second reed switch 4.1 configured to detect the state of the inhalation detection valve 4 is disposed on the wall of the nozzle body 2 adjacent to the second magnet 4.3. The first reed switch 3.1 and the second reed switch 4.1 are connected to a nebulizer control system, wherein the nebulizer control system including an intelligent detection module of the state of the exhalation detection valve and the inhalation detection valve is configured to control an atomizer 5 according to the valve state signal sent by the first reed switch 3.1 and the second reed switch 4.1.

Specifically, the inhalation detection valve 4 is disposed at the bottom of the tubular wall of the nozzle body 2, in which the inhalation detection valve body 4.2 opens upwards as breathing air in from the spray port 2.1; the exhalation detection valve 3 is disposed at the top of the tubular wall of the nozzle body 2, in which the exhalation detection body 3.2 opens upwards as breathing out air to the spray port 2.1. The atomizer 5 could be a vibrating screen atomizer or a mesh type ultrasonic atomizer, wherein in the mesh type ultrasonic atomizer, an ultrasonic mesh atomizing plate vibrates.

If the exhalation detection valve body 3.2 is in an open state, the magnetic sensitive contacts of the first reed switch 3.1 separate; if the inhalation detection valve body 3.2 is in a close state, the magnetic sensitive contacts of the second reed switch 3.1 are being pulled together and connected. If the inhalation detection valve body 4.2 is in an open state, the magnetic sensitive contacts of the second reed switch 4.1 separate, the atomizer 5 is being controlled by the nebulizer control system to start releasing medication; if the inhalation detection valve body 4.2 is in a close state, the magnetic sensitive contacts of the second reed switch 4.1 are being pulled together and connected, the atomizer 5 is being controlled by the nebulizer control system to stop working.

Shown in FIG. 1-3 and FIG. 6 is a nebulizer drug delivery nozzle according to a second embodiment according to the present invention, wherein the structure and arrangement of the nozzle body 2, the exhalation detection valve 3 and the inhalation detection valve 4 could be referred to the description in the first embodiment above. The features of the present embodiment includes: firstly, the first sensor is a first Hall switch instead of the reed switch 3.1 and the second sensor is a second Hall switch instead of the reed switch 4.1, and the first Hall switch and the second Hall switch are connected to the nebulizer control system; secondly, besides the intelligent detection module of the state of the exhalation detection valve and the inhalation detection valve, the nebulizer control system further includes a calculation logic module configured to output the opening angle of the exhalation detection valve 3 and the inhalation detection valve 4. To be specific, the nebulizer control system determines the state of the exhalation detection valve body 3.2 and its opening angle according to the varied magnetic flux of the first Hall switch; if the exhalation detection valve body 3.2 is in a close state, the magnetic flux of the first Hall switch is maximum; if the exhalation detection valve body 3.2 is in a completely open state, the magnetic flux of the first Hall switch is minimum. Similarly, the nebulizer control system determines the state of the inhalation detection valve body 4.2 and its opening angle according to the varied magnetic flux of the second Hall switch; if the inhalation detection valve body 4.2 is in a completely open state, the magnetic flux of the second Hall switch is minimum, the atomizer 5 is being controlled by the nebulizer control system to start releasing medication; if the inhalation detection valve body 4.2 is in a close state, the magnetic flux of the second Hall switch is maximum, the atomizer 5 is being controlled by the nebulizer control system to stop working.

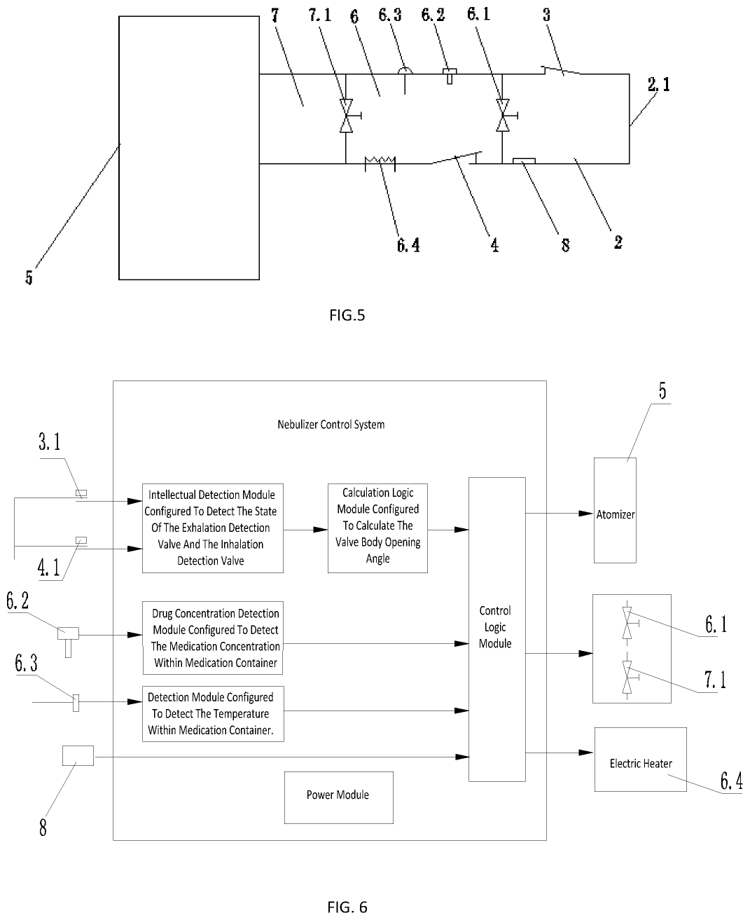

Referring to FIG. 4 and FIG. 6, the nebulizer drug delivery nozzle according to a third present embodiment includes a nozzle body 2 in a shape of tubular, wherein a medication inlet formed at one end of the nozzle body 2 and a spray port 2.1 formed at the other end of the nozzle body 2. In this embodiment, the nebulizer drug delivery nozzle further includes an aerosol container 6.1 which is provided with a drug inlet and an aerosol container valve 6.1. The aerosol container valve 6.2 is a solenoid valve and is connected to the medication inlet of the nozzle body 2. A drug concentration sensor 6.2 is disposed in the aerosol container 6, and the drug concentration sensor 6.2 and the aerosol container valve 6.1 are connected to the nebulizer control system. The nebulizer control system includes an intelligent detection module of the state of the exhalation detection valve and the inhalation detection valve, a valve body opening angle calculation logic module and an inside drug concentration detection module. An exhalation detection valve 3 is disposed at the top of the tubular wall of the nozzle body 2, in which the exhalation detection body 3.2 opens upwards as breathing out air to the spray port 2.1. An inhalation detection valve 4 is disposed at the bottom of the aerosol container 6, in which the inhalation detection valve body 4.2 opens upwards and enters into the aerosol container 6 as breathing air in from the spray port 2.1; The exhalation detection valve body 3.2 is provided with a first magnet 3.3, and the inhalation detection valve body 4.2 is provided with a second magnet 4.3. A first Hall switch 3.1 configured to detect the state of the exhalation detection valve 3 is disposed on the wall of the nozzle body 2 adjacent to the first magnet 3.3, and a second Hall switch 4.1 configured to detect the state of the inhalation detection valve 4 is disposed on the bottom of the aerosol container 6 adjacent to the second magnet 4.3. The first Hall switch 3.1 and the second Hall switch 4.1 are connected to a nebulizer control system. The nebulizer control system determines the state of the exhalation detection valve body 3.2 and its opening angle according to the varied magnetic flux of the first Hall switch; if the exhalation detection valve body 3.2 is in a close state, the magnetic flux of the first Hall switch is maximum; if the exhalation detection valve body 3.2 is in a completely open state, the magnetic flux of the first Hall switch is minimum. Similarly, the nebulizer control system determines the state of the inhalation detection valve body 4.2 and its opening angle according to the varied magnetic flux of the second Hall switch; if the inhalation detection valve body 4.2 is in a completely open state, the magnetic flux of the second Hall switch is minimum, the atomizer 5 is being controlled by the nebulizer control system to start releasing medication; if the inhalation detection valve body 4.2 is in a close state, the magnetic flux of the second Hall switch is maximum, the atomizer 5 is being controlled by the nebulizer control system to stop working.

The intelligent detection module of the state of the exhalation detection valve and the inhalation detection valve and the valve body opening angle calculation logic module in the nebulizer control system output the opening angle according to a one-to-one correspondence between the opening angle and the magnetic flux of Hall switch.

As a preferred manner, the relationship between the opening angle of the exhalation detection valve body 3.2 or the inhalation detection valve body 4.2 and the air flow rate could be obtained by tests based on samples, such that generating a one-to-one correspondence between the opening angle and the air flow rate storing in the nebulizer control system; with which if the nebulizer control system receiving the detected opening angle of the exhalation detection valve body 3.2 or the inhalation detection valve body 4.2, the real-time exhalation air flow rate or the real-time inhalation air flow rate could be determined and prepared for the subsequent atomization process.

The nebulizer drug delivery nozzle according to any one of the above-mentioned embodiments further includes a power module, a nebulizer connection port and a built-in or external user interface, wherein the power module could supply power to each module of the nebulizer control system, so the nebulizer drug delivery nozzle could be used independently.

The nebulizer drug delivery nozzle disclosed in the third embodiment is an intelligent self-adjusting nebulizer drug delivery nozzle, which is adaptive to those manufactured nebulizer for making it more intelligent.

If the drug concentration reaches to an optimal value, the built-in or external user interface could remind the user to inhale and the aerosol container 6 is being opened, in the meanwhile the intake air flow rate and the drug concentration in the aerosol container 6 are being monitored until the drug concentration decrease to below a set value (corresponding to a value that the drug is being completely absorbed), the built-in or external user interface could remind the user of the end of the process. After each time of inhalation, the nebulizer control system calculates the drug amount absorbed, which is the result that the volume of the aerosol container 6 is multiplied by the drug concentration that the user absorbed, wherein the drug concentration that the user absorbed equals to a difference between the drug concentration detected as the user begins to inhale and the drug concentration detected as the user stops to inhale, and accumulates the result. If the accumulation reaches to a necessary amount of treatment, the user is reminded to end the atomization; in this way it could avoid the overdose or deficiency to achieve a precise atomization treatment. Further the efficiency is improved and the treatment time is reduced.

Preferably, before each inhalation, the nebulizer control system could remind the user of holding the spray nozzle 2.1 in mouth and exhaling so as to determine whether the exhalation air flow rate reaches to a preset value; only if the exhalation reaches to a preset value, then opening the aerosol container valve 6.1 so as to ensure a sufficient amount of inhalation.

Referring to FIG. 5 and FIG. 6, on the basis of the nebulizer drug delivery nozzle disclosed in the third embodiment, it further includes an inner medication container 7 provided with an inner medication container valve 7.1 and a nebulizer connection port. The inner medication container valve 7.1 is connected to the drug inlet of the aerosol container 6.

With the inner medication container, the control process of the nebulizer control system includes:

Step 1: detecting the drug concentration within the aerosol container 6 and within the inner medication container 7; if the real-time drug concentration reaches to a preset concentration, firstly closing the inner medication container valve 7.1 to maintain the drug concentration within the aerosol container 6 at an optimal level; reminding the user to inhale.

Step 2: closing the aerosol container valve 6.1 and opening the inner medication container valve 7.1 as the inhalation finishing; then performing step 1 again.

The temperature of aerosol generated in the process of atomization is comparatively low, which may cause reactive airway enthalpy. Hence, the nebulizer drug delivery nozzle may further include a temperature sensor 6.3 and an electric heater 6.4, which may be disposed on the nozzle body 2 or within the inner medication container 7. The temperature sensor 6.3 and the electric heater 6.4 are connected to the nebulizer control system. The nebulizer control system further comprises a temperature detecting module. During the process of atomization, the nebulizer control system automatically regulates the temperature of aerosol according to the detected results so as to ensure the comfort.

An intelligent self-adjusting nebulizer drug delivery device is also disclosed according to another embodiment of the present invention, which comprises a liquid medication container 1, a nebulizer drug delivery nozzle, an atomizer and a nebulizer control system, wherein the nebulizer drug delivery nozzle could be referred to anyone disclosed in the above-mentioned three embodiments.

The intelligent self-adjusting nebulizer drug delivery device may further include an alarm connected to the nebulizer control system. If the nebulizer control system detects the exhalation detection valve 3 and the inhalation detection valve 4 are both in open state at the same time, then closing the atomizer 5 and activating the alarm; the nebulizer control system further includes a sound recognition device, a breathing frequency detection circuit and a voice guide circuit. If the sound recognition device determines the breathing sound is greater than a threshold, or it is determined that the breathing frequency is greater than a preset frequency, the voice guide circuit output an audio instruction.

Compared with the AAD (Adaptive Aerosol Delivery) technology (self-adjusting nebulizer drug delivery device), the intelligent self-adjusting atomizer drug delivery device disclosed by the present invention is more intelligent, and is an innovative intelligent atomization technology (Intelligent Aerosol Delivery), abbreviated as IAD technology.

A method for using the intelligent self-adjusting nebulizer drug delivery device is also disclosed according to another embodiment of the present invention.

The method for using the intelligent self-adjusting nebulizer drug delivery device includes the following steps.

1) Determining whether the inhalation detection valve body 3.2 in an opening state by the nebulizer control system; if the inhalation detection valve body 3.2 is in an opening state, starting inhalation; determining the airflow rate according to the detected opening angle of the inhalation detection valve body 3.2; activating the atomizer 5 by the nebulizer control system if the airflow rate reaches to a preset range.
2) Adjusting atomization parameters, such as medication volume, medication concentration and the like according to types of medication, which could be obtained by medication type detection or user input, inhalation volume and the like.

During the atomization process, if the inhalation volume does not conform certain patterns, (such as the inhalation volume is much greater than the preset volume, the inhalation volume is much less than the preset volume, the inhalation volume fluctuates, the breathing frequency is higher than a preset frequency or the breathing frequency is lower than a preset frequency), the built-in or external user interface could remind the user of adjusting their inhalation volume or breathing frequency; preferably, if the inhalation volume or breathing frequency maintains at an ideal level, the built-in or external user interface could reminder the user to keep their current inhalation volume or breathing frequency. In addition, it may activate some games or contests to make the user more compliant in treatment.

3) Terminating the atomization process by the nebulizer control system if the inhalation airflow rate is lower than the preset range; calculating a total inhalation volume according to the overall time of this atomization process and inhalation volume to determining whether the total inhalation volume is in a preset optimal range; outputting the result via the built-in or external user interface: such that if the total inhalation volume satisfies the treatment requirement, the built-in or external user interface output corresponding signal to remind the user keeping current state; if the total inhalation volume does not satisfy the treatment requirement, the built-in or external user interface output corresponding signal to remind the user adjusting current state.

Further, the nebulizer control system also detects a total inhalation time and a total exhalation time in one atomization process to calculation a breathing ratio, and then determining whether the breathing ratio is in a rational setting range; and outputting the result to user via the built-in or external user interface.

Further, after one atomization process, the nebulizer control system reminds the user of holding their breath for 5 to 10 seconds via the built-in or external user interface and monitors if the user performs corresponding action; if the user does not hold breath, then reminding the user to adjust.

4) Monitoring parameters such as breathing frequency, breathing volume, total breathing volume and breathing ratio in the atomization process; if the breathing begins to present a shallow and frequent pattern, it means the inhalation volume is comparatively low, increasing the time period of atomization process to ensure treatment effect.

Further, the method for using the intelligent self-adjusting nebulizer drug delivery device further includes an adverse reaction detection step and an adverse reaction processing step:

5) If in the atomization process, the breath frequency is higher than a preset adverse reaction threshold, it could be considered as a sign of the occurrence of adverse reaction; determining whether the breathing sound is higher than a preset sound threshold via the sound recognition device of the nebulizer control system; if the breathing sound is higher than the preset sound threshold, terminating the atomization process and reminding the user to exhale forcefully.
6) Compared the exhalation volume within one second as the user to exhale forcefully with a sampled exhalation volume at the beginning of the atomization process; if the two exhalation volumes are significantly different, it is determined as the occurrence of an adverse reaction.
7) If the situations in Step 5 and Step 6 repeatedly occur, terminating the atomization process, generating an adverse reaction report and reminding the user to contact a doctor.

Further, the method for using the intelligent self-adjusting nebulizer drug delivery device further includes a precise medication dosage control step:

8) Gradually increasing the medication concentration within the aerosol container 6 and within the inner medication container 7 from the minimum to the maximum therapeutic concentration via the nebulizer control system as the atomization process starts.
9) if detecting an adverse reaction in the process as gradually increasing medication concentration, then stopping the action of increasing medication concentration and set the current medication concentration as an optimal concentration for the user by the nebulizer control system.

While the invention has been specifically described in connection with certain specific embodiments thereof, it is to be understood that this is by way of illustration and not of limitation. Reasonable variation and modification are possible within the scope of the forgoing disclosure and drawings without departing from the spirit of the invention which is defined in the appended claims. This written description uses examples to disclose the invention, including the best mode, and also to enable any person skilled in the art to practice the invention, including making and using any devices or systems and performing any incorporated methods. The patentable scope of the invention is defined by the claims, and may include other examples that occur to those skilled in the art. Such other examples are intended to be within the scope of the claims if they have structural elements that do not differ from the literal language of the claims, or if they include equivalent structural elements with insubstantial differences from the literal languages of the claims.

1. A nebulizer drug delivery nozzle comprising a nozzle body in a shape of tubular with one end forming a medication inlet and the other end forming a spray port, wherein the nozzle further comprises:
an exhalation detection valve arranged on the tubular wall of the nozzle body, which is configured to open one-way outwards as breathing out air to the spray port, including an exhalation detection valve body provided with a first magnet,
an inhalation detection valve arranged on the tubular wall of the nozzle body, which is configured to open one-way inwards as breathing air in from the spray port, including an inhalation detection valve body provided with a second magnet;
a first sensor configured to detect the state of the exhalation detection valve, which is disposed on the wall of the nozzle body adjacent to the first magnet;
a second sensor configured to detect the state of the inhalation detection valve, which is disposed on the wall of the nozzle body adjacent to the second magnet; and
a nebulizer control system coupled to the first sensor and the second sensor including an intellectual detection module configured to detect the state of the exhalation detection valve and the inhalation detection valve, which is configured to control an atomizer according to the valve state signal sent by the first sensor and the second sensor;
wherein the inhalation detection valve is disposed at the bottom of the tubular wall of the nozzle body, in which the inhalation detection valve body opens upwards as breathing air in from the spray port; the exhalation detection valve is disposed at the top of the tubular wall of the nozzle body, in which the exhalation detection body opens upwards as breathing out air to the spray port.

2. (canceled)

3. The nebulizer drug delivery nozzle according to claim 1, wherein the first sensor is a first reed switch and the second sensor is a second reed switch; if the exhalation detection valve body is in an open state, the magnetic sensitive contacts of the first reed switch separate; if the inhalation detection valve body is in a close state, the magnetic sensitive contacts of the second reed switch are being pulled together and connected; if the inhalation detection valve body is in an open state, the magnetic sensitive contacts of the second reed switch separate, the atomizer is being controlled by the nebulizer control system to start to release medication; if the inhalation detection valve body is in a close state, the magnetic sensitive contacts of the second reed switch are being pulled together and connected, and the atomizer is being controlled by the nebulizer control system to stop working.

4. The nebulizer drug delivery nozzle according to claim 1, wherein the first sensor is a first Hall switch and the second sensor is a second Hall switch; the nebulizer control system includes a calculation logic module configured to output the opening angle of the exhalation detection valve and the inhalation detection valve; the nebulizer control system determines the state of the exhalation detection valve body and its opening angle according to the varied magnetic flux of the first Hall Switch; if the exhalation detection valve body is in a close state, the magnetic flux of the first Hall switch is maximum; if the exhalation detection valve body is in a full open state, the magnetic flux of the first Hall switch is minimum; the nebulizer control system determines the state of the inhalation detection valve body and its opening angle according to the varied magnetic flux of the second Hall switch; if the inhalation detection valve body is in a full open state, the magnetic flux of the second Hall switch is minimum, the atomizer is being controlled by the nebulizer control system to start to release medication; if the inhalation detection valve body is in a close state, the magnetic flux of the second Hall switch is maximum, the atomizer is being controlled by the nebulizer control system to stop working.

5. A nebulizer drug delivery nozzle comprising a nozzle body in a shape of tubular with one end forming a medication inlet and the other end forming a spray port, wherein the nozzle further comprises:
an aerosol container provided with a drug inlet and an aerosol container valve, which is a solenoid valve and is connected to the medication inlet of the nozzle body;
a drug concentration sensor disposed in the aerosol container;
a nebulizer control system coupled to the aerosol container valve and the drug concentration sensor, which includes an intelligent detection module configured to detect the state of the exhalation detection valve and the inhalation detection valve, a calculation logic module configured to calculate the valve body opening angle and a drug concentration detection module configured to detect the medication concentration within medication container;
an exhalation detection valve disposed at the top of the tubular wall of the nozzle body, which is configured to open upwards as breathing out air to the spray port, including an exhalation detection valve body provided with a first magnet;
an inhalation detection valve disposed at the bottom of the aerosol container, which is configured to open upwards as breathing air in from the spray port, including an inhalation detection valve body provided with a second magnet;
a first Hall switch disposed on the tubular wall of the nozzle body adjacent to the first magnet, which is coupled to the nebulizer control system;
a second Hall switch disposed on the bottom of the aerosol container adjacent to the second magnet, which is coupled to the nebulizer control system;
wherein the nebulizer control system determines the state of the exhalation detection valve body and its opening angle according to the varied magnetic flux of the first Hall Switch; if the exhalation detection valve body is in a close state, the magnetic flux of the first Hall switch is maximum; if the exhalation detection valve body is in a full open state, the magnetic flux of the first Hall switch is minimum; the nebulizer control system determines the state of the inhalation detection valve body and its opening angle according to the varied magnetic flux of the second Hall switch; if the inhalation detection valve body is in a full open state, the magnetic flux of the second Hall switch is minimum, the atomizer is being controlled by the nebulizer control system to start to release medication; if the inhalation detection valve body is in a close state, the magnetic flux of the second Hall switch is maximum, the atomizer is being controlled by the nebulizer control system to stop working.

6. The nebulizer drug delivery nozzle according to claim 5, further comprising a power module, a nebulizer connection port and a built-in or external user interface.

7. The nebulizer drug delivery nozzle according to claim 6, further comprising:
an inner medication container provided with an inner medication container valve and a nebulizer connection port configured to connect to the drug inlet of the aerosol container;
a temperature sensor disposed on the nozzle body or within the inner medication container which is coupled to the nebulizer control system;
an electric heater disposed on the nozzle body or within the inner medication container which is coupled to the nebulizer control system;
the nebulizer control system further comprising a detection module configured to detect the temperature within medication container.

8. An intelligent self-adjusting nebulizer drug delivery device comprising a liquid medication container, an atomizer and a nebulizer control system, and a nebulizer drug delivery nozzle according to claim 4.

9. The intelligent self-adjusting nebulizer drug delivery device according to claim 8, wherein further comprising:
an alarm connected to the nebulizer control system;
wherein if the nebulizer control system detects the exhalation detection valve and the inhalation detection valve are in open state at the same time, then closing the atomizer and activating the alarm;
the nebulizer control system includes a sound recognition device, a breathing frequency detection circuit and a voice guide circuit; if the sound recognition device determines that the breathing sound is greater than a threshold, or it is determined that the breathing frequency is greater than a preset frequency, the voice guide circuit makes a sound.

10. (canceled)
